(19) United States
(12) Patent Application Publication
(10) Pub. No.: US 2022/0327953 A1
(43) Pub. Date: Oct. 13, 2022

(54) APPARATUS FOR TREATING POST-TRAUMATIC STRESS DISORDER

(71) Applicant: Frank J. Bourke, Corning, NY (US)

(72) Inventor: Frank J. Bourke, Corning, NY (US)

(21) Appl. No.: 17/728,685

(22) Filed: Apr. 25, 2022

Related U.S. Application Data
(63) Continuation-in-part of application No. 15/142,438, filed on Apr. 29, 2016, now abandoned.
Application No. 15/142,438 is a continuation-in-part of application No. 15/016,627, filed on Feb. 5, 2016, now abandoned.
Application No. 15/016,627 is a continuation-in-part of application No. 13/770,556, filed on Feb. 19, 2013, now abandoned.
(60) Provisional application No. 61/600,174, filed on Feb. 17, 2012.

(51) Int. Cl. G09B 19/00; A61B 5/0205; A61B 5/0533; G09B 5/06

(52) U.S. Cl. CPC G09B 19/00; A61B 5/0205; A61B 5/0533; G09B 5/067; A61B 5/021

(57) ABSTRACT

A method or system by which a clinician can effectively treat the intrusive symptoms of PTSD in fewer than five sessions. The method depends upon a brief elicitation of the trauma memory that makes the memory susceptible to modification. This is followed by the introduction of new or novel information about the remembered event. Because of the labialization induced by the brief exposure, the new information is incorporated into the structure of the memory and the association between the memory and the traumatic feelings are severed. When this is successfully completed, the client is asked to relate the trauma narrative as the clinician assesses it for autonomic responsivity, fluidity and level of detail. If the client shows no discomfort the clinician invites the client to create a new script that eliminates the experience of the trauma, to relive that version in imago, and then to practice the new scenario in the first person in imago.

### CROSS-REFERENCE TO RELATED APPLICATIONS

This application is a Continuation-in-part of U.S. patent application Ser. No. 15/142,438 filed Apr. 29, 2016, which is a Continuation-in-part of U.S. patent application Ser. No. 15/016,627 filed Feb. 5, 2016, which is a continuation-in-part of U.S. patent application Ser. No. 13/770,556 filed Feb. 19, 2013, which claims priority to U.S. Provisional Patent Application Ser. No. 61/600,174 filed Feb. 17, 2012, the entirety of which is incorporated herein by reference.

### BACKGROUND OF THE INVENTION

The invention is a psychotherapeutic intervention for the treatment of PTSD and other trauma reactions. It may be considered a behavioral intervention in the category of Trauma Focused Cognitive Behavioral Interventions.

PTSD is a debilitating mental illness that impacts the lives of countless Veterans, active combat service members, Veterans, first responders, crime victims and others who have been exposed to life threatening or horrifying circumstances.

All of the front line behavioral interventions, despite having been accorded the status of evidence-based treatments fall far short of expectations. Consistent research has shown that all of these behavioral interventions are equivalent in results (Steenkamp & Litz, 2013; Steenkamp, Litz, Hoge, & Marmar, 2015) and the results, while better than nothing are not impressive (Bisson & Andrew, 2007; Goodson et al. 2011; Steenkamp & Litz, 2014).

Of the two treatments reviewed by Steenkamp and associates (2015), both obtained clinically significant reductions in symptom ratings (10-12 points; Monson, Gradus, et al., 2008; Schnurr, Friedman, et al., 2003) for both self-report and physician administered measures. Both interventions tended to lose symptom reductions at follow-up. Loss of diagnosis, when reported ranged from 17% to 39% of cases).

A 2013 Cochrane systematic review of PTSD treatments for adults (Bisson, Roberts, Andrew, Cooper, & Lewis, 2013) examined 70 studies involving a total of 4761 individual participants. Their analysis found that trauma-focused cognitive behavioral therapies (TFCBT), including PE and CPT, were better than waitlist and treatment as usual controls. The mean severity of symptoms typically dropped by 1.6 s standard deviations.

Standard behavioral treatments for PTSD are largely dependent upon extinction protocols that make use of various exposure-based methods. Such treatments have several drawbacks. All or nearly all require adjunctive or follow-up treatment (Foa, et al., 2000). All risk retraumatization of the patient and minimally require some significant level of re-exposure to the traumatizing event. Insofar as they are extinction-based, positive treatment results tend to fade over time as the hallmarks of the extinction process spontaneous recovery, contextual renewal, reinstatement, and rapid reacquisition assert themselves (Bouton, 2004; Rescorla, 1988; Schiller, Kanen, LeDoux, Monfils, & Phelps, 2013).

The invention described herein departs from previous art in that it is rooted in a neurological mechanism known as reconsolidation blockade and relies upon empirical observation, not abstract theory.

An initial pre-pilot study of the invention was completed in October 2014 with 96% of subjects no longer meeting diagnostic criteria for PTSD. Remediation of intrusive symptoms of PTSD in fewer than five sessions: A 30-person pre-pilot study of the RTM Protocol. Journal of Military, Veteran and Family Health, 1(2), 85-92. doi:10.3138/jmvfh.3119

A second, unpublished pilot study of the RTM intervention, more rigorous than the first, was recently completed in San Diego, Calif. That study, a 30 person waitlist controlled protocol with blinded psychological measures, replicated the findings of the earlier study.

An earlier search of the journal literature for memory mechanisms that explain the pattern observed with the protocol found that the reconsolidation mechanism, was the only pattern of application and efficacy that matched the reported results. Although reconsolidation has been observed in all memory systems, the following information is focused upon its application to emotional memories (Gray & Bourke, 2015).

Reconsolidation describes the reactivation of long term, otherwise permanent memories, by their evocation in certain contexts. When a memory is reactivated, it becomes subject to change. If the circumstances surrounding the memory remain the same, the memory remains unchanged; it reconsolidated and is maintained in its current state. If circumstances have intensified, the impact of the memory may become worse; retraumatization can add to the intensity of trauma memories; the reconsolidation process allows complimentary information to be added to the memory. If the new circumstance provides evidence that the threat predicted by the trigging stimulus (CS) is no longer relevant, the strength of the affective charge may decrease; the new information blockades the emotional response to the memory and adds in the new information. New information, relevant to the original memory, especially when novel, supports reconsolidation. Without new, relevant information, memory updating through the reconsolidation mechanism does not occur. New information produces a so-called prediction error. A prediction error is a presentation that violates expectations about the circumstance predicted by the stimulus. This may be created by not requiring a complete retelling of the trauma, telling it in a new circumstance, voluntarily telling the story, and/or manipulating the structure of the experience. The introduction of new information blockades the reassertion of the old memory structure and transforms it.

This invention, the Reconsolidation of Traumatic Memories (RTM) protocol is a brief treatment that is often completed in six sessions or fewer during which, the clinician follows a set procedure in a safe setting (Gray & Bourke, 2015). The procedure starts with a brief, controlled reminder of the trauma that, according to reconsolidation theory, renders the traumatic memory subject to change. The reminder of the traumatic memory is interrupted and never allowed to bloom into a full, traumatic, re-experiencing event. After a period of ˜10 minutes, the old memory enters a period of malleability (lability) in which it becomes subject to change. This so-called ‘window of lability’ may last as long as six hours. The procedure then adds in dissociative experiences which, it is believed, transform the affective response to the memory. After treatment, the memory is accessible but non-traumatizing. Unlike extinction-based protocols, the disturbing symptoms are not subject to spontaneous recovery, contextual renewal and other extinction phenomena; the symptoms disappear and stay away. After treatment, the traumatic event becomes available to non-traumatizing declarative access. Clients typically and spontaneously reassess the meaning of the event and reintegrate the memory into their life narrative.

### SUMMARY OF THE INVENTION

It is the object of the present invention to provide a new, brief, clinician-guided, non-traumatizing intervention for the elimination of the intrusive symptoms of PTSD. It is estimated that 50 to 75% of all PTSD patients suffer from this variety of the disorder.

The invention is distinct from other therapies aimed at the disorder in that it is a neurological intervention, rooted in the syntax of memory reconsolidation blockade and deals directly with the neurological structure of the remembered trauma. While the visual and dissociative formats employed by the intervention may be altered (as detailed in TABLE N), the reconsolidation format underlying the visual and dissociative specifics is critical to their formulation and the protocol's efficacy.

The mechanism upon which the invention relies is distinct from extinction, the neurological process upon which PTSD therapies generally judged to be most effective are based in that it is not subject to spontaneous recovery, contextual renewal and the other hallmarks of extinction learning.

The invention is distinct from other interventions for PTSD in that it adheres closely to the pre-clinical syntax of reconsolidation blockade including: 1) identification of a target, previously existing, trauma memory; 2) a brief non-reinforced or interrupted reminder of that target memory; 3) a pause or distraction; 4) the introduction of new, relevant, or novel information conceived as changes in the structure of the formatting of the visual, kinaesthetic and auditory elements of the trauma memory; 5) following the intervention the memory is retested to assess its transformation or lack thereof.

The invention is also distinct from other PTSD interventions in that clients who complete the intervention typically and spontaneously reappraise the meaning of the memory, find an appropriate context for the memory, gain new perspective about the memory, and reintegrate the traumatic memory, all without specific guidance from the therapist.

The invention requires the guidance of a trained clinician because there is potential for adverse emotional reactions and the process is generally too complex to be managed without guidance. More specifically, the clinician is needed in order to: 1) guide the client through the intervention process; 2) assess autonomic reactivity at various points in the intervention; and 3) provide a safe means of freeing the client from overwhelming responses should they occur.

The main phases and components of the present invention are the following:

1) The client identifies a most important or most significant index trauma related to current experiences of nightmares, flashback and intrusive thoughts. This is typically the one most closely related to the content of the flashbacks and nightmares that are the central symptoms for the client. In cases where there is a series of events or the index trauma extends over a long time period, the client is encouraged to identify the single, central, or most important incident.

2) The intervention may be continued either with full disclosure of the content or as a content free intervention.

3) The clinician asks the client to relate the trauma narrative as fully as possible, or to imagine encountering the kind of event that triggers a typical flashback. In many cases the client will be so overcome with emotion and automatic negative responses that they cannot relate the entire story.

4) The clinician must observe the client closely and as soon as the client begins to show signs of autonomic arousal (while relating the event), he is to stop the retelling and reorient the client to the present time and context.

5) The clinician now asks the client to describe two neutral events, one from just before the traumatic event (neutral start) and a second from after the event, when the client knew that event itself was over, and that he or she was safe and alive (neutral end). The client is then asked to imagine entering a movie theater where the neutral start picture is on the screen in black and white.

6) After choosing a seat and sitting down, the client is to imagine floating up out of their body and into the projection booth from which they can see their body in the theater and the screen displaying the B&W neutral beginning picture.

7) At the clinician's instruction the client is to watch their own body in the theater as they watch a black and white movie of the trauma event on the movie screen. The movie begins with the neutral start image and ends with the neutral end image. After running the movie, the client is asked to rate their comfortability with the movie process.

8) If there was discomfort the black and white movie is rerun with modifications as needed (as described in the DETAILED DESCRIPTION, Table 5, below) until the B&W movie produces no discomfort.

9) When the movie scenario feels comfortable, client is invited (on cue) to float back down into their own body and walk to the movie screen where they will step into the neutral end picture (still on the screen), restore color and sound to the image and, in about two seconds, relive the entire scenario fully associated, in reverse, experiencing the event undoing itself from within, ending at the safe, neutral beginning picture (Neutral start). This may be repeated with adjustments (as described in DETAILED DESCRIPTION below) until it can be completed comfortably.

10) Client is now asked to relate the trauma narrative. When the narrative can be repeated with little or no impact, as if at a distance with increased detail, the process continues to the next phase. If there is still strong autonomic responsivity, the entire movie sequence may be repeated.

11) Client is now invited to design a new version of the trauma scenario in which either they were not injured, different choices were made, or the event did not occur.

12) When a satisfactory exemplar has been constructed, the client imagines going through it in first position, as if they were actually living through it.

13) If a first run through of the revised scenario is not acceptable, a new scenario may be generated. When a satisfactory scenario has been identified, it may be repeated six to eight times.

14) Client is now asked to relate the trauma narrative. When the trauma narrative can be repeated without discomfort, as if at a distance with increased detail, the process is presumed to be over. If there is still strong autonomic responsivity, the rescripted scenario, or a new rescripted scenario may be repeated with a different set of modifications.

While this specific visual/dissociative sequence has been developed as a highly effective generalization, successful administration sometimes necessitates alteration of the specific components while adhering to the underlying reconsolidation format. These and further details and alternative presentations are presented in the detailed description below.

### DETAILED DESCRIPTION

Inclusion and Exclusion Criteria

The method described defines specific inclusion and exclusion criteria:

It is essential that the client's difficulties are essentially a phobic, instantaneous conditioned response to the narrative of a traumatic event that threatened immanent death or injury to self or others, an element of that narrative, or a stimulus related to the involuntary evocation of the aforesaid traumatic event.

Where multiple traumas are involved each must be separable from the others as a distinct event. That is: each event must have an identifiable starting and end point even if they belonged to the same broad emotional, geographic or temporal context.

The problem must 1. Be rooted in the personal experience of trauma threatening death or injury to one's self or others, and 2. Be expressed as an intense, suddenly arising experience of the trauma symptoms usually experienced as flashbacks or panic reactions.

Additionally, the central symptoms for the appropriate use of the protocol are recurrent flashbacks (at least once a month) and nightmares (at least once a month). If these intrusive elements are missing, the protocol is inappropriate.

Where such nightmares and or flashbacks are present, the intervention is appropriate when, in the course of describing the index trauma, or when exposed to a triggering stimulus, the client displays signs of autonomic arousal which may include but are not limited to, freezing, flushing, shaking, sweating, changes in facial color, involuntary motor responses, changes in breath rate, etc. In addition, while relating a trauma narrative, such arousal may be reflected by pauses, loss of detail, loss of fluidity, inability to speak, pressured speech, etc. (see Table 1, below).

The traumatic response must be in regard of a specific event; it may not be a response to the meanings of the event in the client's larger life or the impact of such events on the client's sense of self-worth. Such excluded problems may include, but are not limited to, grief, guilt, shame, anxiety, and anger.

If both kinds of response are present, the client must be advised that the protocol will work for the intrusive elements with an immediately arising phobic-like response. RTM will eliminate flashbacks and other immediate panic responses to reminders of the traumatic event—but may have no effect on larger life issues which will require other kinds of treatment.

While the invention targets the intrusive symptoms of PTSD, its use may be associated with the ending of other co-occurring problems which are secondary responses to the nightmares and flashbacks themselves. Such relationships have been observed with substance use disorders, hypervigilant, and avoidant symptoms; nevertheless, these will not be affected in all cases.

Rapport

The therapeutic perspective adopted in the RTM process assumes that change work occurs in the subjective space between two or more people. It also assumes that each person has a distinctive representation of the world that does not in all points correspond either to another person's representation of reality or to the common reality with which they must deal. In order to communicate across this representational gap and to establish a common set of understandings for such communication, it is important for the clinician to understand and at least provisionally accept the clients understanding of the world and of their current problem. This acceptance is facilitated by the understanding that the method here describes deals with the subjective structure of the problem, not its content. Rapport is an essential tool in this regard.

Rapport may be understood as the establishment of a state of trust or empathy between persons. It may also be understood as a state of mutual sensitivity to the meanings and values held by the participants in the communication.

Rapport may be established by matching the client's use of language predicates and their paralinguistic behaviors. Specific techniques may include matching posture, breathing, predicates, voice tone, rate and volume, etc. In general, however, presenting an open and honest concern for the client's outcome, and carefully eliciting feedback to ensure that you have correctly understood their meaning usually produces the most significant level of rapport. Rapport is usually manifested by a dance of matched movement, breathing and other paralinguistic elements as the participants consciously or unconsciously exchange nonverbal signals (Bandler & Grinder, 1979, Gray, 2011). In this context, rapport does not guarantee self-disclosure, only cooperation with the treatment.

Rapport may also be crucial for trauma work as it can provide a significant link to the present as a safe place or context in case the elicitation of the problem state becomes too intense. Insofar as a strong rapport has been established, it serves as a positive physiological reference point for the RTM process itself. The establishment of rapport may be considered a conditioned response to a positive present state elicited by the practitioner's physical presence. In such cases the client can be called back into the present moment by commenting on something, the request that they look the practitioner in the eye and breathe with them, or some other reminder of the present.

Framing the Invention

Framing provides a meaningful context for the intervention. It sets up specific expectations about the procedure, gains crucial permissions and is used to defuse any anxiety associated with treatment. In practice, it allows the practitioner to set the limits on discomfort that the client may experience and serves to review the general procedures to be used. Framing also serves to separate the RTM intervention from previous forms of treatment that may have been unpleasant or ineffective. It does so by discussion the distinctive features of the invention as contrasted with treatments that are ineffective or employ techniques that include the possibility of retraumatization through exposure to the index trauma in vivo, in silico, or in imago. In doing so, it serves to eliminate anxieties related to reliving the trauma or losing control. Finally, the frame is important in establishing a relationship in which the client is empowered to stop and question the procedure as necessary.

The Framing Procedure:

Explain that you will be doing a short visualization process that is ordinarily comfortable, but sometimes has a very short period of moderate discomfort.

Assure the client that at the first signs of discomfort, you will stop the procedure and make adjustments as necessary.

Ask about any previous therapy or attempted interventions, and explain how the process you will use is very different, in particular, explain that RTM does not involve “reliving” the traumatic events through exposure or “catharsis” or “release” of feelings.

Then ask, “Do you have any questions or concerns before we begin?” If the client has any concerns about doing this process, respond to them congruently, and assure them that if any questions or concerns arise at any time during the process, it is fine to interrupt it and to tell you what they are.

As part of the framing process, before the active intervention begins, have the client practice the central parts of the intervention with one or two neutral, pleasant, or innocuous experiences.

There are at least two parts of the intervention that are sufficiently complex that they often require that the client practice them before proceeding. This complexity and the need for providing guidance to the client is one of the reasons that a trained clinician is essential to the process.

The two elements that typically require practice are the black and white dissociated movie and the fully associated rewind.

Practice

Explain to the client that she will be doing some things in her imagination that she may never have done before and that it would be useful to have some practice. After obtaining her consent, ask the client to think of a neutral or mildly negative experience. Have them identify a place or time just before the identified scene clearly begins (neutral start) and a place or time where the scene has clearly ended (neutral end). For example, for a scene representing a memory of eating lunch, the start image might be walking through the doorway into the kitchen. The end scene, might represent putting the dishes in the sink. Each scene clearly marks a point outside of the event described as ‘eating lunch.’ The neutral start and end images are to be held as still, black and white images that signal the boundary of the target memory. Have the client practice viewing the experience from beginning to end as a black and white movie projected on a wall or screen outside of themselves. Each iteration of the practice movie begins with the still, neutral start image and ends with the still neutral end image. Do this until the client can perform the task with ease. Similarly, after stopping the black and white movie, have the client practice stepping into the end frame of the black and white movie (neutral end), turning on the colors and sounds and running the entire scenario backwards as an imaginal, present time, fully-associated experience of the same movie, lasting only about two seconds. Have the client practice these two elements, one-at-a-time until they are done easily and then proceed with the treatment.

Elicitation of the Traumatic Response

According to Foa and colleagues (Foa, Keane, & Friedman, 2000; Foa and Kozak, 1986; Foa & Meadows, 1997), elicitation of the problem state is the sine qua non of effective trauma treatment. They also warn specifically against the use of ineffective probes or questioning techniques that fail to elicit a valid physiological experience of the trauma response. The RTM protocol relies on multiple steps, and the assessment of physiological concomitants of the client's response at each step. A simple verbal answer regarding the problem state is never sufficient; the practitioner must be able to identify the specific signals of the fear response as they arise in the present time (See Table 1.). If there is no physiological response indicating that the phobic response has been triggered, the intervention will not be successful. Likewise, at the end of the protocol it is important to verify that the fear response is no longer generated in response to the previous triggers.

Although the preferred method for using the intervention is to elicit some kind of description of the traumatic incident, this intervention may be pursued content free. That is, the practitioner does not need to know the specific content of the presenting problem, and the questions don't need to be answered verbally; they are only asked in order to elicit the physiological changes, and to verify that these are immediate and intense. However, brief content information about triggers can be useful at the end of the protocol to verify that the client has a neutral response to them.

Trauma Narrative

Accordingly, after establishing rapport, the practitioner should ask about the problem state in order to elicit the physiological response. The following kind of question is suggested, allowing only enough of a response to elicit the problem response:

What is the worst time that you had your problem response? (flashback, panic, etc.) When and where does this typically occur? (crowded rooms, open spaces, etc.) What triggers your response? (Humvees, loud noises, Arab clothing, etc.)

More simply, the clinician may ask: “Tell me what happened?”

As the client responds, the practitioner must attend to the physiological and paralinguistic elements that reflect heightened arousal and the elicitation of the problem response. One of the most reliable signs of having identified an appropriate trigger is the fast onset of the physiological and paralinguistic symptoms of fear or trauma. Table 1 lists these symptoms.

TABLE 1
Signs of Autonomic responsivity comprising:

Changes in breath rate
Changes in heart rate
Changes in muscular tension
Changes in skin color
Changes in skin tone
Changes in voice pitch
Changes in speech rate
Flushing
Freezing
Involuntary motor responses
Loss of detail in narrative
Loss of fluidity in narrative
Mutism
Pauses in narrative
Postural changes
Pressured speech
Shaking
Sweating
Tremors

It may be useful to make notes as to what the client was saying as the symptoms began, whether they were focused inwardly or outwardly, or any other changes observed.

A response to these probes, by an appropriate client, usually results in a narrative that is truncated, lacking details, characterized by short vignettes punctuated by strong autonomic arousal. It may be impossible for the client to complete the narrative.

These responses serve several essential purposes. 1. They allow the clinician to clearly identify the problem response in terms of its physiological expression and to verify that it is an appropriate phobic response that arises quickly and intensely. 2. They allow the clinician to identify the specific trigger or stimulus that evokes the state in terms of the client's own descriptive behavior at the time of symptom onset. 3. By providing a typical response to the eliciting stimulus, they provide information that can be used to identify the problem state if it re-emerges in the testing phase after a failed or incomplete treatment. 4. The absence of this response to triggers in the testing phase will signal the success of the treatment. 5. According to reconsolidation theory, the process cannot begin until a short reminder stimulus (enough to awaken autonomic responsivity) has been elicited.

Eliciting the nonverbal problem response engages the practitioner and client in a reciprocal relationship that arouses the autonomic system, sensitizing the client to the relevant stimuli. The active participation of the client in consciously accessing the problem state begins the cognitive restructuring of the problem state using the technique of prescribing the symptom. The deliberate elicitation of the flashback or phobic state represents the reminder phase of the reconsolidation process and represents a novel context that facilitates the labilization of the trauma memory. By actively participating in eliciting the problem state, the client gains an experience of control over what was previously an uncontrollable and unpredictable traumatic response. This is also an example of the therapeutic use of Bandura's concept of self-efficacy in the context of PTSD treatment.

The literature of Reconsolidation indicates that reconsolidation only occurs when the target memory is aroused in a novel context or a novel manner; if there is no new information, the memory will not become subject to updating.

Break State

From time to time, especially during the recitation of the trauma narrative and sometimes during other parts of the protocol, clients will become associated into a negative experience and begin to show signs of autonomic reactivity such that they are appear to moving towards a full flashback or abreaction. In such cases, it is essential, as soon as the first signs of arousal appear (Table 1) to stop the progress of the experience. The options used to break state are listed in Table 2 Breaking State. Other examples are available in the literature are listed in Table 2.

TABLE 2
Break State Patterns Comprising:

Distracting the client so as to return them to a neutral or resourceful state
by asking the client about their favorite music, favorite foods, where they
grew up, or any other innocuous topic
Orienting the client to a different sensory system (looking at a view of
nature, something in the room, listening to music or a sound)
Physically moving the client to a different position: e.g., walking around
the room
Picking up a conversation that ended before initiation of treatment as if
treatment never happened
Having the client become aware of their feet on the floor and their
presence in the room.
Looking the client in the eye while calling their name and instructing
them to be present

The Movie Theather Scenario

Ask the client if they've ever been to a movie theater. Request that they recall a familiar one and recall how it was decorated, how it was arranged, the colors employed, how it smelled, etc.

Inform the client that you will be using the movie theater as part of the intervention. In a few minutes, they will be watching themselves, watching themselves as they watch a black and white movie of the problem event. But that is not going to happen yet.

Help the client to establish neutral start and neutral end points for the index trauma memory. Remind them that you are not going to delve into the memory but you just need to know what they were doing before it started and how they knew that the event itself was over using language comprising: What were you doing then? Find the neutral end point by asking (e.g.), “How did you know that this event was over? Just this moment, this event. What did you see and hear that let you know that it was done?”

Similarly, for the neutral start image (e.g.): “What were you doing just before this happened? Perhaps it was something completely different.”

Have the client imagine the neutral start and neutral end images as still pictures that will bookmark the beginning and end of the black and white movie.

Ask the client to imagine walking into that theater where there is, displayed on the screen, a black and white, still picture of the neutral start image. Have them describe the physical aspects of the theater comprising: whether there is a stage, a proscenium arch, curtains, the colors of the interior, the color of the chairs, the fabric of the chairs, whether the floor is carpeted?

Instruct the client to walk half to three quarters of the way towards the rear of the theater to find a seat that they find comfortable. Ask them to describe the seat and its environs using questions comprising whether it folds or slides, whether it is adjustable or not, what the fabric is, whether it is comfortable, whether the floor is sticky or not, what the arm rests feel like, etc.

Once the client is comfortably seated, remind them to notice the still black and white picture on the screen. Have them notice that it represents a still picture from the time before anything happened.

The Black and White Movie

Instruct the client to listen to the following instructions first and then to complete them on cue.

Instruct the client to imagine themselves floating up out of their own body, there in the theater, to a place in the projection booth, above and behind them. In the projection booth they are to walk up to and place their hands on a thick old, plexiglass window that looks out over the theater. Instruct them to appreciate the thickness of the window, to notice whether there is yellowing or scratches on it, and to look down into the theater to see their own body, down there, in the theater, looking at the black and white picture projected on the screen. Tell the client to do this as you repeat the instructions. Have them relax in their chair and watch their own body, there in the theater, looking at the still picture on the screen.

Once the client is situated in the projection booth, they should exhibit signs of dissociation comprising the elements listed in table 3.

TABLE 3
Signs of Dissociation comprising:

Client leans back in chair (they are not leaning forward; they are not
pointing or gesturing)
Head is back, chin is up
Posture is open (it is not folded in, or protective)
Facial expression is relaxed and symmetrical (there are no signs of
concentration or effort)
Muscles are relaxed (there is no obvious tension, they are not
holding on to themselves or the furniture)
Breathing is measured
Pulse is regular and even

If the projection scenario does not provide an adequate level of dissociation as indicated by the elements comprising those listed in Table 3, other means may be used to enhance the client's level of dissociation from the theater, his alternate position in the theater, and the still picture on the screen comprising those listed in Table 4.

TABLE 4
Methods Used to Enhance the Initial Dissociation in the Movie Theater: From
time to time it will become necessary to provide alternate means to assert
an appropriate level of dissociation in the client. This may be especially
necessary during the first part of the procedure in which the client may
have difficulty dissociating from his image in the theater, the neutral
start image or the basic black and white move. The methods, ordered by frequency
of use and from the least intrusive to the most, comprise:

Moving the client from the projection booth to a ‘meta’, angelic, or divine
perspective above the theater and instructing them to watch themselves in the
projection booth as they watch the self in the theater as they watch the movie.
With and only with the client's explicit permission, gently pulling them back into
an upright position in the chair by gently tugging on their hair.
With and only with the client's permission, creating an anchor or conditioned
stimulus for the dissociative state by using a distinctive yet familiar comforting
touch to keep them mindful of a safe, dissociated state.
Similar modifications and distortions may be included to ensure dissociation
from the movie contents.

Once a satisfactory level of dissociation has been attained, instruct the client to listen to the following instruction but not to execute them until given the cue.

On cue, but not before, the client is to remain in the projection booth with their hands on the glass looking down at their alternate self in the theater (or other dissociative positions as suggested in Table 4), turn on the black and white movie, and watch themselves (down there in the theater), as they watch the black and white movie from beginning to end; starting with the neutral start image and ending with the neutral end image. At the end of the movie, the neutral end image is to remain on the movie screen.

It is important to emphasize to the client that they are not to watch the movie itself. They are only to watch their alternate self, down there, in the theater, as they watch the movie. The client should pay special attention on how the person, down there in the theater, responds. It may be useful to emphasize that there is no connection between the observer in the projection booth and the watcher in the theater. For all intents and purposes, they are unrelated.

When the client is ready, have them start the movie, reminding them to focus upon the person in the theater and their responses, as the movie runs.

When the client completes a run-through, whether or not there is evident autonomic arousal, the clinician should ask the client, how the segment was for him and for the watcher in the theater. If either the client (in the projection booth) or the watcher in the theater experienced any discomfort, the movie should be repeated or adjusted as follows.

During the running of the black and white movie, the clinician is to observe the client for any of the signs of autonomic arousal as noted in Table 1. Mild arousal may be tolerable but at any point where the client becomes clearly upset, or if the client asks to stop, the clinician is to stop the procedure, break state using the methods described in table 2 (if necessary).

Once the client is restored to the present context with no signs of continuing arousal, the clinician should negotiate, with the client's full participation, an alternate presentation of the black and white movie using techniques comprising those listed in table 5.

TABLE 5
Methods Used to Enhance Dissociation: From time to time it will become necessary
to provide means to assert or reassert a level of dissociation in the client.
This may be especially necessary during the first part of the procedure
in which the client may have re-associated into the experience. of the
black and white move (described below). The methods, ordered by frequency
of use and from the least intrusive to the most, comprise:

| | |
|---|---|
| Moving the movie screen farther away | Moving the client from the projection |
| from the watcher's seat in the theater | booth to a ‘meta’, angelic, or divine |
| Turning the movie screen or moving | perspective above the theater and |
| the watcher's seat (down in the | instructing them to watch themselves in the |
| theater) so that it is only visible at an | projection booth as they watch the self in |
| oblique angle. | the theater as they watch the movie. |
| Allowing the movie to be displayed as | With and only with the client's explicit |
| first only the bottom half of the | permission, gently pulling them back into |
| screen, beginning to end, and then the | an upright position in the chair by gently |
| top part of the screen, beginning to | tugging on their hair. |
| end | With and only with the client's permission, |
| Playing every odd numbered second | creating an anchor or conditioned stimulus |
| of the movie, beginning to end, | for the dissociative state by using a |
| followed by every even numbered | distinctive yet familiar comforting touch to |
| second of the movie beginning to end. | keep them mindful of a safe, dissociated |
| If there is sound (there is usually | state. |
| none), muffling, deadening or | Other similar modifications and distortions |
| distorting the sound so that it is no | may be included to ensure dissociation |
| longer bothersome. | from the movie contents. |
| Instead of using the projection booth | |
| scenario, having the client stand | |
| behind their own body in the theater, | |
| with their hands on their other selves' | |
| (the watcher's) shoulders, while | |
| attending to that other selves' | |
| response to the black and white movie | |

Having agreed on such a modification, the movie scenario is repeated (with more adjustments if needed) until the black and white movie can be completed with no discomfort. When the client completes the black and white movie without incident it may be repeated several times before proceeding to the associated rewind. Each time that the movie is completed, at the end of the movie, the neutral end image is to remain on the movie screen.

Associated Reversal

Once the client is comfortable with the black and white movie, instruct the client to listen to the following instruction but not to execute them until given the cue.

Float down from the projection booth and back into your body down there in the theater. After fully associating into your body, get up from your seat, walk to the isle and then walk down the aisle to the movie screen where the black and white, neutral end image is still displayed.

Go ahead and do that now.

instruct the client to listen to the following instructions but not to execute them until given the cue.

When I give you the instruction, step into the neutral end picture, turn on the colors and the sound, and the feeling, and experience the entire event moving backwards from the neutral end image to the neutral start image, all in about two seconds. Illustrate with a right to left gesture of the hand (if sitting beside the client) while making a sound like, “Bezzouuuuurrrrppppp!”

Impress upon the client that they are to experience the entire event as if they were experiencing it in reverse: everything is undoing itself; sounds run backward, motions are reversed, everything reverses its normal order. The scenario ends with the client associated into the neutral start image.

When the client indicates their understanding, tell them to step into the neutral end picture, turn on the colors, sounds and motions, and rewind the event.

During the running of the reversed associated movie, the clinician is to observe the client for any of the signs of autonomic arousal as noted in Table 1. Mild arousal may be tolerable but at any point where the client becomes clearly upset, or if the client asks to stop, the clinician is to stop the procedure and break state using the methods described in Table 2 (if necessary).

Once the client is restored to the present context with no signs of continuing arousal, the clinician should ask them about their experience, and identify any problematic parts of the reversed movie.

If there are significant problems with associating into the event at all, it may be necessary to redo the black and white movie scenario with new adjustments to ensure the client's comfort. If there are mild problems, including brief pauses, or brief indicia of autonomic arousal it may be useful to repeat the reversal several times with specific instructions for seeing a motion (s) or action(s) in reverse, hearing a sound(s) in reverse, or other kinds of associated sensory reversals.

Once the client is restored to the present context with no signs of continuing arousal, the clinician should ask them about their experience of the reversal. If no problems are reported the procedure continues with the narrative.

Trauma Narrative

Ask the client to tell you the story of the index trauma, beginning with the neutral start place, continuing through the event and ending with the neutral end scene.

If the narrative is completed without the signs of autonomics arousal detailed in Table 1, this segment of the intervention is deemed complete. If a significant level of arousal remains, the clinician must decide whether to repeat the entire procedure from the Movie theater scenario for ward, or just the rewind.

When the narrative can be completed as a distant event, without autonomic arousal, or only minor arousal, proceed to the rescripting phase.

Rescripting

After the client has successfully retold the trauma narrative without significant arousal, he may proceed with the rescripted narrative.

Invite the client to create a new or novel scenario for the trauma memory in which (despite beginning at the same neutral start): a) they were not injured; b) different choices were made that changed the nature of the incident; c) the event was a movie, play or charade of some kind; or d), the event did not occur. Allow the client to design an alternate scenario that is acceptable to them.

Once an acceptable scenario has been designed, have the client imagine going through it, fully present and associated: as if this was the way the event happened. Remind them that this is just a fantasy, and the truth remains the same, but that it will be useful to experience the event in this new way.

Have the client run through the new version of the event once and check with them to ensure that it works for them. If the new scenario works for them, If the scenario needs changing, work with the client to create a more acceptable version. Have the client run through it once, and if satisfactory, have the client repeat it four to eight times.

If the rescripted scenario can be repeated without problems, move to the narrative.

Narrative

Ask the client to tell you the story of the index trauma, beginning with the neutral start place, continuing through the (actual) event and ending with the neutral end scene.

If the narrative is completed without the signs of autonomic arousal detailed in Table 1, the intervention is deemed complete. If a significant level of arousal remains, the clinician must decide whether to repeat the entire procedure from the Movie theater scenario forward, or just a new scenario.

When the narrative can be completed as a distant event, without autonomic arousal, full of detail, often accompanied by an ‘aha’ experience, the procedure is presumed to be over.

In the new method a galvanic sensor is worn inside a wrist band and monitored during the therapy session to confirm to the patient is entering into a “panic” state. The panic state measures whether the patient is experiencing stressed breathing, or heart irregularity that shows a heightened level of stress. These collections of real time patient monitoring are the patient's real time physiological settings. They can be used in conjunction with a real time recording of the patient/therapist recording to identify potential stress memories that are part of a target trauma.

The therapist starts to direct the patient to disassociate from a troubling memory by cuing the patient to recognize they are in a theater setting rather than directly in the stressful setting that invokes anxiety and a panic response.

The therapist then directs the patient to a coded safe setting from a dialog that was recorded with the patient to identify that state. The dialog is indexed to a recorded and identified low stress setting that the patient can view upon being chosen and display by the therapist.

It has been found the therapy works more effectively when objective measurements signal the therapist to start a disassociation protocol with the patient. In a preferred system the apparatus has recording capabilities that record by code a target trauma memory, a safe memory and a safe background that a recording of the patient inserted into the safe background.

The safe background with patient recording can be displayed by the therapist to the patient and is coded so that the safe designation and an identifying key phrase guides the therapist in displaying the safe background to the patient.

In physical form the safe background can be an audiovisual recording of a segment of a recording with the therapist that has as a segment of the recording a steaming record of the patient's that has been identified as a post target trauma safe memory to help disassociate the patient from the target trauma.

The patient/therapist discussion follows the general process outlined above and is recorded with the concurrent patient physiological record. The physiological record is classified as safe, stressful or traumatic by identification by the patient and the therapist with a rating input device starting with the rapport section of the therapist/patient discussion and following with the discussion of the therapist direction to imagine a safe setting, such as a theater. The stress rating continues with the further steps and forms a basis for providing stress level criteria during the therapy.

The coding device can be a simple dial labelled with stress levels. Or a squeeze device that measures grip that each person, the patient and the therapist, use where more pressure indicates more stress

The advantage of the use of the device is to identify patient stress level to the therapist.

What is claimed is:

1. A system for treating PTSD comprising:
A recording system for recording conversations between a therapist and a selected PTSD patient and records live conversations.
A device that measures blood pressure, heart rate, and galvanic change continuously that is worn by the patient and displays on the live conversations,
A set of stress factor limits for said patient indicating safe, moderately stressful, and potentially traumatic stress levels; and
A library of safe background recordings of the patient.

2. The apparatus of claim 1 further comprising A) a recording of said PTSD patient, B) the recording of establishing rapport with the client, including physiological measurements of said client C) the recording of discovering the index trauma of said client including said physiological measurements of said, D) identifying and separating discrete traumas of said client, E) eliciting from said client and terminating a brief re-experience of the index trauma, F) guiding the client through the treatment, G) evaluating levels of autonomic arousal of said client, H) preventing retraumatizations and abreactions by said client, I) determining which modifications to the intervention are appropriate, and J) the recording of testing for completion of treatment. wherein the clinician guides the client through the steps comprising: a) Identifying the target trauma; K) Eliciting and stopping the trauma narrative; L) Guiding practice; M) Establishing distinct start and end points for the index trauma by applying alarm limits of said physiological record of said N) Creating imaginal movie theater and showing patient the recorded safe background; O) Establishing dissociation; P) Presenting the black and white movie of the recording of the index trauma

3. The apparatus of claim 1 wherein said apparatus further comprises: recordings of said safe background that keeps said patient from retraumatizations and abreactions by said patient, applying alarm signals for heightened physiological alarms to trigger showing said safe background.

4. The apparatus of claim 4 said safe background of A) a movie screen; B) a wall; C) the inner eyelids; D) other imagined projection surface.
